(19) United States
(12) Patent Application Publication
(10) Pub. No.: US 2024/0065630 A1
(43) Pub. Date: Feb. 29, 2024

(54) ORTHOPEDIC BRACE FEATURES INCLUDING MEASURING PIVOT ANGLE

(71) Applicant: flashBrace LLC, St. Paul, MN (US)

(72) Inventors: Michael Kane, St. Paul, MN (US); Mark Holten, St. Paul, MN (US); Cullen Holten, St. Paul, MN (US)

(21) Appl. No.: 18/239,245

(22) Filed: Aug. 29, 2023

Related U.S. Application Data
(60) Provisional application No. 63/402,363, filed on Aug. 30, 2022.

(51) Int. Cl. A61B 5/00; A61B 5/11; A61F 5/01

(52) U.S. Cl. CPC A61B 5/6812; A61B 5/0004; A61B 5/1121; A61F 5/0102; A61B 2505/09; A61B 2562/0219; A61B 2562/0223

(57) ABSTRACT

Systems and methods for implementing orthopedic braces and appliances are described, and in particular measuring a pivot angle between components such as for creating enhanced functionalities that improve the user experience, comfort, and device acceptability while augmenting the rehabilitation value of orthopedic braces.

### PRIORITY APPLICATION

This application claims the benefit of priority to U.S. Provisional Patent Application Ser. No. 63/402,363, filed Aug. 30, 2022, the content of which is incorporated by reference in its entirety.

### TECHNICAL FIELD

This document pertains generally, but not by way of limitation, to systems and methods for implementing orthopedic braces and appliances, and in particular measuring a pivot angle between components such as for creating enhanced functionalities that improve the user experience, comfort, and device acceptability while augmenting the rehabilitation value of orthopedic braces.

### BACKGROUND

Current statistics for the United States indicate that musculoskeletal injuries account for 77% of all injury health care visits and result in $176.1 billion U.S. dollars of treatment costs per year. In addition to injuries, osteoarthritis, rheumatoid arthritis, and traumatic arthritis conditions cause substantial rates of joint surgeries. These include surgical repair and joint replacements. In the United States, approximately 600,000 knee replacement procedures and 450,000 hip replacement procedures are performed each year. The medical treatment of orthopedic injuries and rehabilitation of patients is frequently assisted by means of external orthopedic appliances commonly referred to as braces. Though the exact number of orthopedic braces prescribed after injuries and surgeries is not known precisely, 17.1 million sprains, 18.3 million fractures, 17.7 million “other” musculoskeletal injuries and over 1 million joint replacement surgeries (USA) per year are indicative of a very substantial demand for rehabilitation appliances.

There are many brace types currently in common use. The functional premise of these devices is to support tissues and to control the range of motion of the affected anatomy. These actions can help speed recovery, prevent re-injury, and enable safe mobility by limiting movement. Design and construction variations can include soft, rigid, and flexible materials in various combinations to effect substantially rigid to substantially flexible implementations. The braces can include high levels of sophistication in materials, fitting adjustments, stabilizing elements, pivots, hinges, and some include adjustable range of motion features.

The various braces reflect their myriad functional and therapeutic objectives. Pre-surgical immobilization, post-surgical immobilization, progressive rehabilitation, injury recurrence prevention, and pain management are but a few of the many purposes served. The affected joints and limbs are as diverse as the range of human injuries and physical degradations. All of the moveable joints of the body are potential injury or disease loci, though some are substantially more likely to be problematic. The most common orthopedic maladies of the moveable joints affect the knee, hip, elbow, wrist, ankle, shoulder, back and neck.

While some medical procedures such as spinal and ankle fusion are intended to permanently limit motion, the vast majority of orthopedic surgical interventions endeavor to preserve, improve, or restore musculoskeletal functionality. After medical interventions, the therapeutic trajectory is frequently directed towards an incremental transition from protective stabilization to maximally achievable functional mobility.

### SUMMARY/OVERVIEW

A characteristic of many rehabilitative, post-surgical, and chronic-support brace devices is the use of hinged or flexural elements. The functional premise of these devices is to support tissues and to control the range of motion of the targeted anatomy. Some devices allow for limited or adjustable range of motion around a specific axis or within a fixed plane. Many of these are configured to permit the gradual or incremental return of normal anatomical function by supporting safe exercise modes for the wearer.

The restorative power of modern orthopedic interventions, surgical and non-surgical, is amplified by the sophistication of physical rehabilitation techniques. The targeted, incremental, and controlled exercise challenges to the recovering patient have a profound impact on the speed and depth of recovery. Some of these challenges occur with the direct supervision and assistance of skilled physical therapists, physical therapy technicians, and their allied professionals. However, in most outpatient recovery situations, the patients must conduct the majority of their rehabilitation exercises without aid, at home, or in their own environments. Left to their own devices and motivations, many patients simply lose interest or motivation to perform their exercises. Many do not wear their braces as prescribed for a variety of reasons. “Adherence to home exercise in rehabilitation is a significant problem, with estimates of nonadherence as high as 50%, potentially having a detrimental effect on clinical outcomes.

The present inventors have recognized, among other things, that a problem to be solved can include failure to adhere to rehabilitation potentially having a detrimental effect on clinical outcomes for a significant portion of patients requiring rehabilitation. The present device or techniques can help provide a solution to this problem, such as by helping to improve the functional scope, enhance the social acceptability, and increase the pleasure and comfort of orthopedic devices and braces.

The present disclosure generally relates to systems and methods for implementing orthopedic braces and appliances with enhanced functionalities that improve the value and utility of orthopedic braces. The range of the implementations contemplated in this disclosure can include modules that append to existing brace devices to enhance aesthetic value, modules that can append to existing braces to enhance functional utility, integrated brace systems with improved functionality elements incorporated that augment the standard design, integrated appliances with improved functionality modules or aesthetic elements that are conceived in the original appliance design but remain optional for the user to add after the acquisition of the basic brace.

This Summary/Overview is intended to provide an overview of the device or techniques of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components.

The drawings illustrate generally, by way of example, but not by way of limitation, various embodiments discussed in the present document.

FIG. 1 is a perspective view of an example of an orthopedic brace affixed to a patient's leg.

FIG. 2 is a diagram of an example of a bracing portion of an orthopedic brace.

FIG. 3 is a diagram of an example of a bracing portion of an orthopedic brace with an integrated range of motion sensor with a coaxial magnetic field.

FIG. 4 is a diagram of an example of an orthopedic brace with an integrated range of motion sensor with a non-coaxial magnetic field.

FIG. 5 is a cross section of an example of a magnet embedded in a brace member.

FIG. 6 is a cross section of an example of an orthopedic brace with an integrated range of motion sensor.

FIG. 7 is a diagram of an example of an embedded hub-magnet configuration.

FIG. 8 is a diagram of an example of an embedded hub-magnet configuration.

FIG. 9 is a diagram of an example of an embedded hub-magnet configuration.

FIG. 10 is a diagram of an example of an embedded hub-magnet configuration.

FIG. 11 is a diagram of an example of an embedded hub-magnet configuration.

FIG. 12 is a diagram of an example of an embedded hub-magnet configuration.

FIG. 13 is a diagram of an example of an embedded hub-magnet configuration.

FIG. 14 is a diagram of an example of an embedded hub-magnet configuration.

FIG. 15 is a diagram of an example of an embedded hub-magnet configuration.

FIG. 16 is a diagram of an example of an embedded hub-magnet configuration.

FIG. 17 is a diagram of an example of an embedded hub-magnet configuration.

FIG. 18 is a diagram of an example of an embedded hub-magnet configuration.

FIG. 19 is a diagram of an example of an embedded hub-magnet configuration.

FIG. 20 is a diagram of an example of an embedded hub-magnet configuration.

FIG. 21 is a cross section of an example of an external magnet affixed to a brace member with a clip.

FIG. 22 is a diagram of an example of a bracing portion of an orthopedic brace configured with an external magnet.

FIG. 23 is a diagram of an example of an orthopedic brace member with an added function board.

FIG. 24 is a diagram of an example of an orthopedic brace member with an added function board.

FIG. 25 is a perspective view of an example of an adapter clip.

FIG. 26 is a perspective view of an example of an aesthetic cover.

FIG. 27 is a diagram of an example of a bracing portion of an orthopedic brace with an integrated gyroscopic range of motion sensor.

FIG. 28 is a diagram of an example of a bracing portion of an orthopedic brace with an integrated gyroscopic range of motion sensor.

FIG. 29 is a diagram of an orthopedic brace being used in conjunction with external exercise or other training equipment.

FIG. 30 is a block diagram of an example of an apparatus, device, or machine upon which any one or more of the techniques (e.g., methodologies) discussed herein may be performed.

### DETAILED DESCRIPTION

This document pertains generally, but not by way of limitation, to systems and methods for implementing orthopedic braces and appliances, and in particular measuring a pivot angle between components such as for creating enhanced functionalities that improve the user experience, comfort, and device acceptability while augmenting the rehabilitation value of orthopedic braces. A variety of orthopedic braces may be used to help achieve a variety of therapeutic objectives.

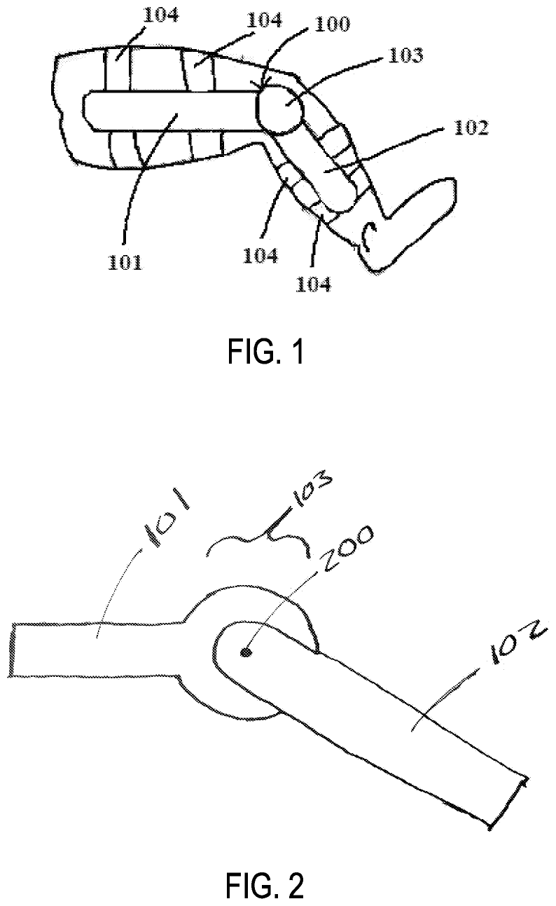

FIG. 1 shows an example of an orthopedic brace 100. The orthopedic brace 100 may include a bracing portion. The bracing structure of an orthopedic brace 100 may include a first brace member 101, a second brace member 102, and a hub 103. The first brace member 101 and the second brace member 102 may connect and rotate around the hub 103, such as by using a hinged, flexural, or other connection that allows these components of the orthopedic brace 100 to rotate with respect to a pivot point 200. An orthopedic brace 100 can be configured to be externally affixed to a patient's leg, such as by using straps 104 or other attachment technique.

FIG. 2 depicts an example of a bracing portion of an orthopedic brace 100. The orthopedic brace 100 may include a first brace member 101, a second brace member 102, and a pivot point 200 about which the orthopedic brace 100 may rotate. The connection of the first brace member 101, and the second brace member 102 may be referred to as a hub 103. As a part of therapy for a patient with a brace, it may be desirable to track the motion of the orthopedic brace 100 around the hub 103, such as to track a patient's range of motion. The motion of an orthopedic brace 100 may be monitored, such as can include using a system of magnets and sensors.

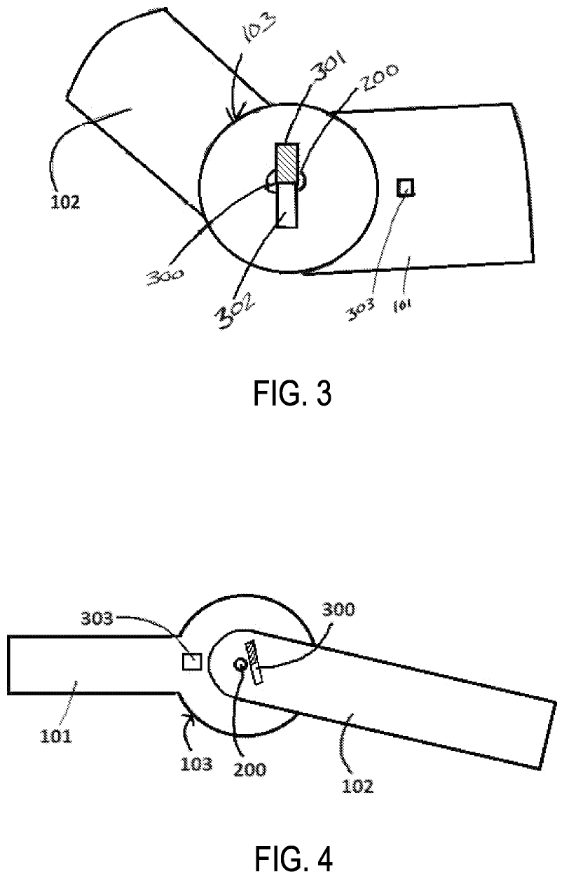

FIG. 3 shows an example of a close up of a hub 103 of a bracing portion of an orthopedic brace 100. The hub 103 can include a magnet 300 with a North pole 301 and a South pole 302 that can be affixed to, or embedded in, the hub 103. The magnet 300 in FIG. 3 can be configured to produce a magnetic field that is coaxial with the pivot point 200. A magnetic field sensor 303 can be affixed to or embedded in the second brace member 102 and configured to monitor the magnetic field produced by the magnet 300, such as by measuring the magnetic vector at the location of the magnetic field sensor 303. In configurations in which the magnet 300 produces a magnetic field that is coaxial with the pivot point 200, the magnetic field can be described as a function of an angle θ of the orthopedic brace 100, such as a linear distribution. The angle θ is defined by the angle between the first brace member 101 and the second brace member 102 about the pivot point 200. When the magnetic field as a function of angle produces a linear distribution, no corrections are needed. In such a case, an orthopedic brace 100 may include circuitry configured to compare a magnetic vector (V x) collected by the magnetic field sensor 303 at angular intervals. The circuitry can determine the change in position of the orthopedic brace 100. This can be described in an equation as:

$$\frac{d θ}{dt} = \frac{{V}_{2} - {V}_{1}}{{t}_{2} - {t}_{1}}$$

$$or$$

$$Δθ = {V}_{2} - {V}_{1} .$$

In practical cases, a magnetic field may not provide a linear distribution. This can be due to factors such as an off-axis magnet 300 or magnetic field sensor 303, field curvature, ferromagnetic materials, a combination of those factors, or other factors that may impact the magnetic field. FIG. 4 shows an example of a bracing portion of an orthopedic brace 100 with a hub 103 that includes a magnet 300 that can be configured to produce a magnetic field that is not coaxial with the pivot point 200. The angular position of an orthopedic brace 100 over time in a case where the magnetic field central axis is not substantially coaxial with the pivot point 200 of the orthopedic brace 100 can be determined, and this determination can include correcting for the nonlinearity of the distribution. The magnetic field sensor 303 can include compensation circuitry configured to correct for a non-linear field distribution.

Examples of a magnetic field sensor 303 can include, but are not limited to, a magnetometer, a Hall sensor, or a giant magnetoresistance effect (GMR) sensor. The magnetic field sensor 303 can include a single-axis magnetic field sensor 303 or a multi-axis magnetic field sensor 303. An orthopedic brace 100 with a single-axis magnetic field sensor 303 may account for the nonlinearity with a calibration data set that can be collected, such as B=f(θ)″, for θ₁, θ₂, . . . θₙ where B can be a scalar value of the sensed field and θ is the angular position of the orthopedic brace 100. The calibration data set can then be used to create a lookup table such as the following example:

| | |
|---|---|
| B₀ | θ₀ |
| B₁ | θ₁ |
| Bₙ₋₁ | Θₙ₋₁ |
| Bₙ | θₙ |

In practice, a lookup table can include or consist of around 12 pairs of angles and scalar values for a 110-degree arc. The lookup table may have more or less pairs of angles and scalar values. More pairs may help increase accuracy. Much fewer than 12 pairs may reduce accuracy. An orthopedic brace 100 can then be configured to calculate the angular position of the orthopedic brace 100 for values of B that fall between values Bₓ and Bₓ₋₁/collected for the lookup table by interpolation between θ(Bₓ) and θ(Bₓ₊₁). A correction for a non-linear field distribution can include fitting the values of 0 and B from the calibration data set to an equation. Equations with better fit quality may help keep computational error low when calculating derivative functions. Many functions can be used for the equation. Polynomials can be practical and fast in microprocessor implementations. Linear equations and quadratic equations may have poorer fit, so polynomials of third order or higher may be better options.

A multi-axis magnetic field sensor 303 can be advantageous over a single-axis magnetic field sensor 303, such as by helping to discriminate movements for extended mobility joints such as a hip, spine, neck, or shoulder joint. A multi-axis magnetic field sensor 303 can be used with an orthopedic brace 100 that can include a pivot point 200 configured as a bearing type pivot, such as a ball and socket, hinge and axle, or distributed flexural link. A multi-axis magnetic field sensor 303 can produce a combination of Bx, By, and Bz signal. A two argument arctan function can be used to process two signals, such as Bx and By, to produce a result where θ=F(Bx, By). A two argument arctan function can be corrected for non-linearity with respect to joint angle. The result can be used in combination with the interpolation or equation techniques described above. A two argument arctan function can help eliminate ambiguity or discontinuity of a tangent function and can help allow the data to be used for 360 degrees of arc.

### Magnet Configurations

Additional examples of possible magnet configurations can include a magnet 300 that can be affixed to either the first brace member 101 or the second brace member 102 with the magnetic field sensor 303 affixed to the other of the first brace member 101 or the second brace member 102. The magnet 300 and magnetic field sensor 303 can be embedded or affixed externally such as with a clip.

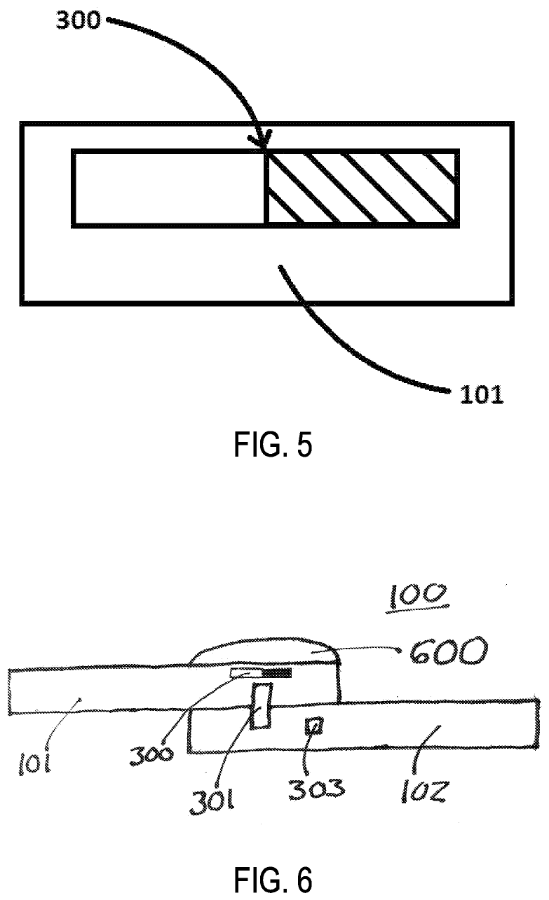

FIG. 5 shows an example of a cross section of a magnet 300 embedded in a brace member 101. Embedding the magnet 300 can offer the advantage of being an economical way to incorporate a magnet 300 into the design of the pivot point 200 of many modern orthopedic braces 100, such as by embedding the magnet 300 into a receiving cavity in the non-ferrous structure of the brace frame. Embedding the magnet 300 in non-ferrous structural components of a brace can help integrate the magnet 300 into the mechanism of a brace joint without significant change to the thickness of the structures, which can help include the magnet 300 without affecting the joint operation. An embedded magnet 300 may also be less visually obtrusive, and embedding a magnet may help to prevent damage, dirt buildup, or disturbances to the magnet from external sources.

Longer magnets can provide better pole separation than shorter magnets, which can in turn produce a superior magnetic field shape. Embedding a magnet 300 in a brace member can compromise the strength of the brace member where material has been removed to create a receiving cavity for the magnet. However, an embedded magnet that crosses less than 90% of the width of the brace member can result in acceptable magnetic fields while retaining sufficient residual brace member strength. Placing the magnet 300, closer to the magnetic field sensor 303 can help produce superior magnetic field strength which can help make readings more reliable. A thin magnet 300, such as a magnet 300 with a thickness of less than 2 mm, can be desirable for embedding in an available space of an existing pivot point 200. Desirable examples of magnets can be disk magnets, toroidal or washer magnets, or flat magnets with high length or width to thickness ratios, such as a ratio greater than 6. One practical example of a desirable magnet 300 can be a magnet 300 approximately 0.5 mm thick with a length or width of around 8 mm or greater.

FIG. 6, which depicts a cross section of an example of a bracing portion of an orthopedic brace 100 and a brace monitoring system that can include a first brace member 101, a second brace member 102, a pivot point 200, a magnet 300 and a magnetic field sensor 303 as well as a hub cover 600. A hub cover 600 can be advantageous as the hub cover 600 can help protect a magnet 300 placed in the hub 103 area. A purpose of the hub cover 600 may be to help conceal the pivot point 200 or to help protect the user, such as from pinching due to pivoting. The hub cover 600 can help protect the magnetic field sensor 303. Generally, the magnet 300 embedded in the hub 103 interacts with a magnetic field sensor 303 on the hub 103 surface or in the hub 103 body. Other magnetic materials can be included, such as to help shape, capture, or focus, magnetic flux lines.

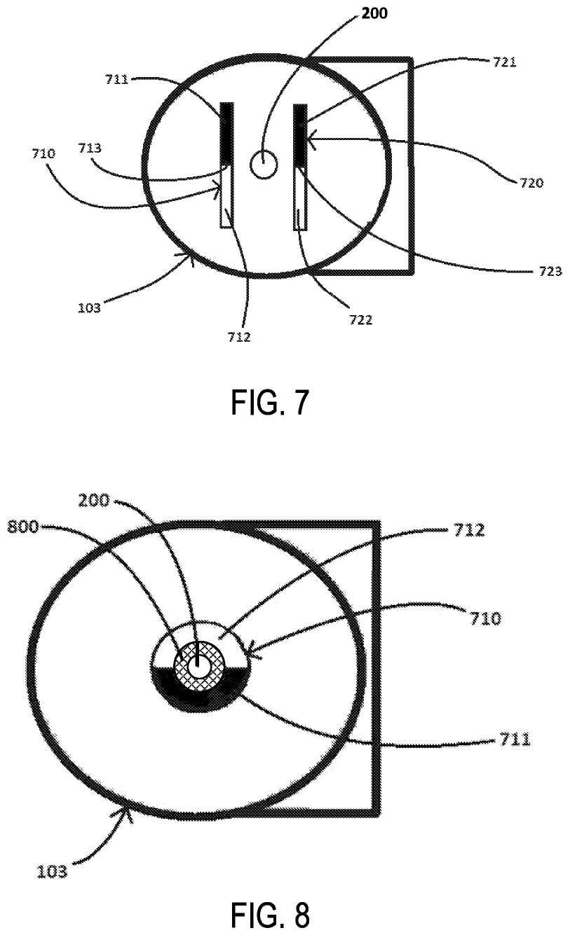

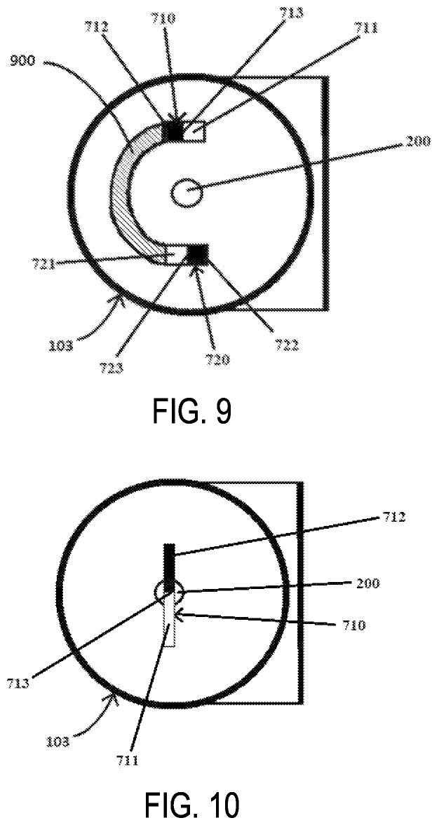

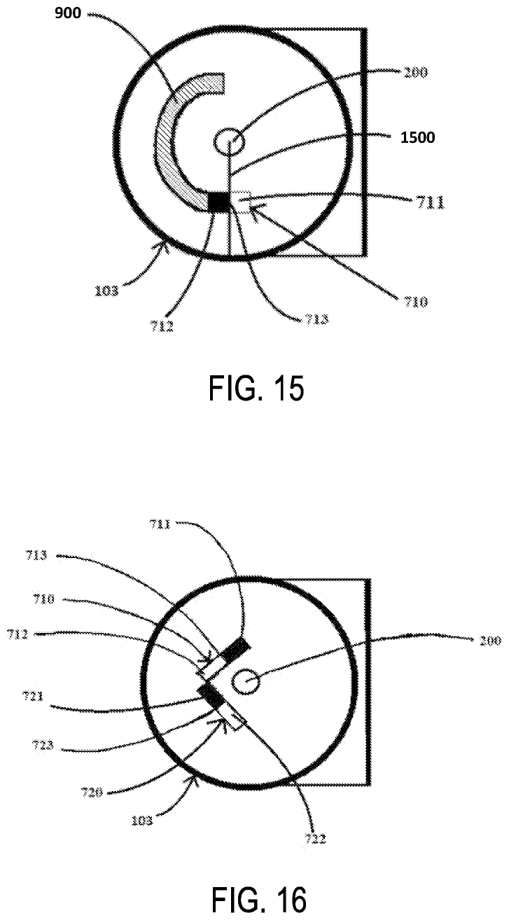

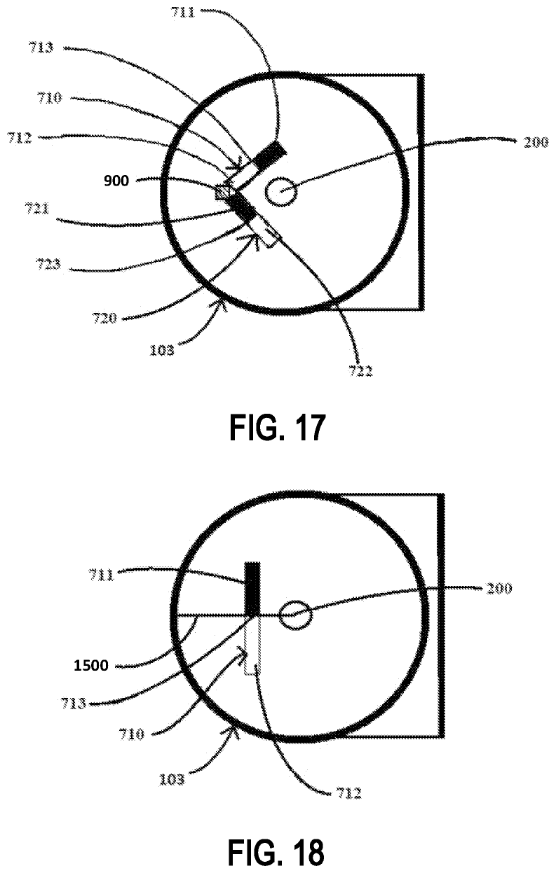

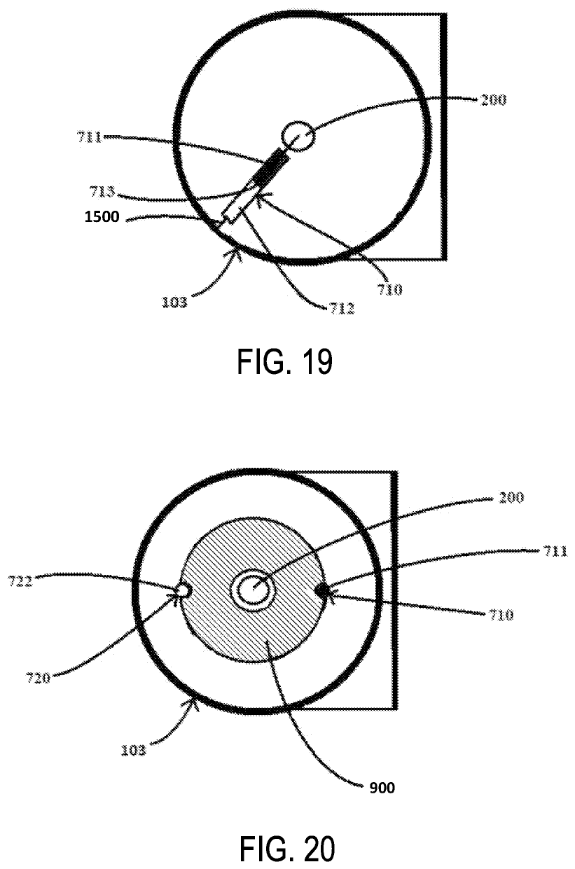

FIGS. 7-21 show examples of configurations in which one or more magnets 300 can be embedded in the hub 103 of an orthopedic brace 100 such that the one or more magnets produce a magnetic field that can be monitored by a magnetic field sensor 303 to help determine the pivot angle of the orthopedic brace 100. Configurations with external magnets are also possible, but may not be as effective. The examples of configurations shown in FIGS. 7-21 can include a hub 103, a pivot point 200, and one or more magnets 300. The configurations shown in FIG. 8, FIG. 9, FIG. 12, FIG. 14, FIG. 15, FIG. 17, and FIG. 20 can also include a ferromagnetic conductor 900, such as soft iron.

FIGS. 7-16 show examples of configurations that can produce a magnetic field that can be coaxial with the pivot point 200 of the orthopedic brace 100, and FIGS. 18-19 show configurations that can produce magnetic fields that may be substantially coaxial with the pivot point 200 of the orthopedic brace 100, but may produce magnetic fields that can be slightly asymmetric.

FIG. 7 depicts an example of a hub 103 of an orthopedic brace 100 that can be configured with a first magnet 710 having a first magnetic pole 711, a second magnetic pole 712, and a midpoint 713, and a second magnet 720 having a first magnetic pole 721, a second magnetic pole 722, and a midpoint 723. For configurations with more than one magnet, the first magnetic pole 711 of the first magnet 710 and the first magnetic pole 721 of the second magnet 720 can either both be a North pole, or both be a South pole with the second magnetic pole 712 of the first magnet 710 and the second magnetic pole 722 of the second magnet 720 both being the opposite polarity of the first pole. In FIG. 7 the first magnet 710 and the second magnet 720 can include substantially parallel straight bar magnets that can be embedded in the hub 103 such as on opposite sides of the pivot point 200. The first magnet 710 and second magnet 720 can be configured to produce a magnetic field with a center that is coaxial with the pivot point 200 of the hub 103 to be monitored by the magnetic field sensor 303.

FIG. 8 depicts an example of a hub 103 of an orthopedic brace 100 that can be configured with a first magnet 710 having a first magnetic pole 711, and a second magnetic pole 712, and a bearing 800. In FIG. 8, the first magnet 710 can include a substantially circular disk magnet embedded in the hub 103 forming a ring with a center at the pivot point 200 of the hub 103. A bearing 800, such as a nylon, or other plastic, bushing, can fill the space between the pivot point 200 and the interior boundary of the first magnet 710. The bearing 800 can help protect the first magnet 710 and pivot point 200 from abrading each other. The combination of the first magnet 710 can be configured to produce a magnetic field that is coaxial with the pivot point 200 to be monitored by a magnetic field sensor 303.

FIG. 9 depicts an example of a hub 103 of an orthopedic brace 100 that can be configured with a first magnet 710 having a first magnetic pole 711, a second magnetic pole 712, and a midpoint 713, and a second magnet 720 having a first magnetic pole 721, a second magnetic pole 722, and a midpoint 723. In FIG. 9 the first magnet 710 and the second magnet 720 are substantially parallel straight bar magnets embedded in the hub 103 on opposing sides of the pivot point 200 and connected by a C-shaped ferromagnetic conductor 900. The combination of the first magnet 710 and ferromagnetic conductor 900 can be configured to produce a magnetic field that is coaxial with the pivot point 200 to be monitored by a magnetic field sensor 303.

FIG. 10 depicts an example of a magnet configuration that can include a first magnet 710 with a first magnetic pole 711, a second magnetic pole 712, and a midpoint 713. In FIG. 10, the first magnet 710 is a straight bar magnet embedded in the hub 103 with a midpoint located at the pivot point 200 of the hub 103 and can be configured to produce a magnetic field that is coaxial with the pivot point 200 to be monitored by a magnetic field sensor 303.

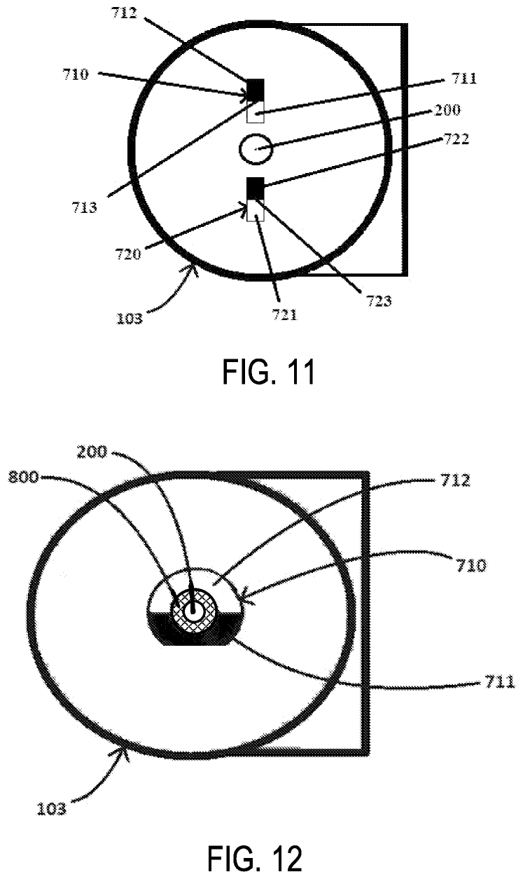

FIG. 11 depicts an example of a hub 103 of an orthopedic brace 100 that can be configured with a first magnet 710 having a first magnetic pole 711, a second magnetic pole 712, and a midpoint 713, and a second magnet 720 having a first magnetic pole 721, a second magnetic pole 722, and a midpoint 723. The first magnet 710 and the second magnet 720 can be substantially colinear (axially aligned) straight bar magnets embedded in the hub 103 on opposing sides of the pivot point 200. The first magnet 710 and second magnet 720 can be configured to produce a magnetic field with a center that is coaxial with the pivot point 200 of the hub 103 to be monitored by a magnetic field sensor 303.

FIG. 12 depicts an example of a hub 103 of an orthopedic brace 100 that can be configured with a first magnet 710 having a first magnetic pole 711, and a second magnetic pole 722, and a bearing 800. In FIG. 8, the first magnet 710 is a disk magnet, having a flat side and otherwise circular shape, embedded in the hub 103 forming a ring with a center at the pivot point 200 of the hub 103. A bearing 800 such as a nylon, or other plastic, bushing, can fill the space between the pivot point 200 and the interior boundary of the first magnet 710. The bearing 800 can help protect the first magnet 710 and pivot point 200 from abrading each other. The combination of the first magnet 710 and can be configured to produce a magnetic field that is coaxial with the pivot point 200 to be monitored by a magnetic field sensor 303. The flat side of the first magnet 710 can help to align the first magnet 710 with the orthopedic brace 100 which can help to simplify manufacturing.

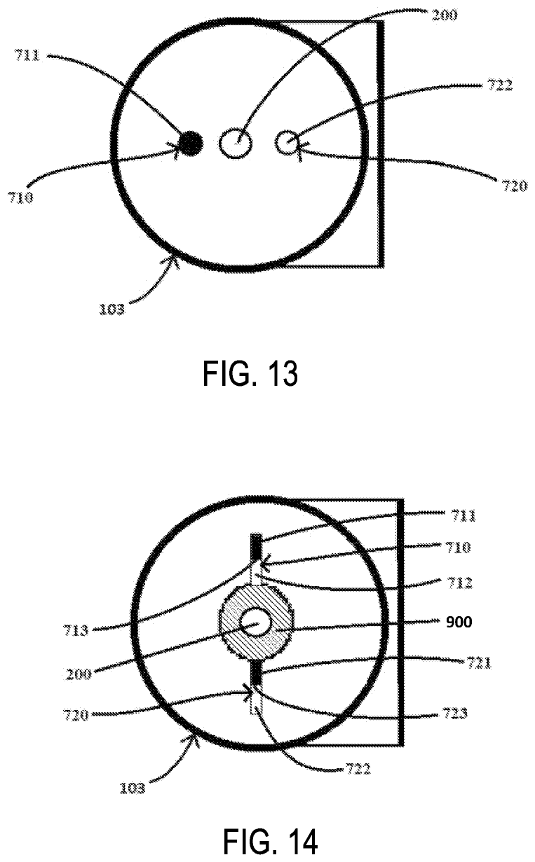

FIG. 13 depicts an example of a hub 103 of an orthopedic brace 100 that can be configured with a first magnet 710 having a first magnetic pole 711, a second magnetic pole 712, and a midpoint 713, and a second magnet 720 having a first magnetic pole 721, a second magnetic pole 722, and a midpoint 723. The first magnet 710 and second magnet 720 can be embedded in the hub 103 on opposing sides of the pivot point 200 such that the first magnetic pole 711 and second magnetic pole 712 of the first magnet 710 are divided along a plane perpendicular to the axis of rotation and the first magnetic pole 721 and second magnetic pole 722 are also divided along a plane perpendicular to the axis of rotation and such that first magnetic pole 711 of the first magnet 710 and the second magnetic pole 722 of the second magnet 720 face the same direction. The first magnet 710 and second magnet 720 can be configured to produce a magnetic field with a center that is coaxial with the pivot point 200 of the hub 103 to be monitored by a magnetic field sensor 303.

FIG. 14 depicts an example of a hub 103 of an orthopedic brace 100 that can be configured with a first magnet 710 having a first magnetic pole 711, a second magnetic pole 712, and a midpoint 713, and a second magnet 720 having a first magnetic pole 721, a second magnetic pole 722, and a midpoint 723, and a ferromagnetic conductor 900. The first magnet 710 and the second magnet 720 can be substantially colinear straight bar magnets embedded in the hub 103 on opposing sides of the pivot point 200. A ferromagnetic conductor 900 can connect the first magnet 710 to the second magnet 720. The ferromagnetic conductor 900 can be ring shaped with a center at the pivot point 200. The ferromagnetic conductor 900 can be configured in a “C” shape, or a variety of other open or closed track shapes. The first magnet 710, second magnet 720, and ferromagnetic conductor 900 can be configured to produce a magnetic field with a center that is coaxial with the pivot point 200 of the hub 103 to be monitored by a magnetic field sensor 303.

FIG. 15 depicts an example of a hub 103 of an orthopedic brace 100 that can be configured with a first magnet 710 having a first magnetic pole 711, a second magnetic pole 712, and a midpoint 713, and a ferromagnetic conductor 900. The first magnet 710 can be a straight bar magnet configured to be offset from the pivot point 200 and perpendicular to a radius 1500 of the hub. The ferromagnetic conductor 900 can be configured as a semi-circle extending around the pivot point 200 with one end connecting to the first magnet 710. The first magnet 710 and ferromagnetic conductor 900 can be configured to produce a magnetic field with a center that is coaxial with the pivot point 200 of the hub 103 to be monitored by a magnetic sensor 303.

FIG. 16 shows an example of a hub 103 of an orthopedic brace 100 that can be configured with a first magnet 710 having a first magnetic pole 711, a second magnetic pole 712, and a midpoint 713, and a second magnet 720 having a first magnetic pole 721, a second magnetic pole 722, and a midpoint 723. The first magnet 710 and second magnet 720 can be straight bar magnets offset from the pivot point 200 and positioned such that the second magnetic pole 712 of the first magnet 710 meets the first magnetic pole 721 of the second magnet 720 such that the first magnet 710 and second magnet 720 form an angle that opens toward the pivot point 200. The first magnet 710 and second magnet 720 can be configured to produce a magnetic field that is substantially coaxial with the pivot point 200 of the hub 103 to be monitored by a magnetic field sensor 303.

FIG. 17 shows an example of a hub 103 of an orthopedic brace 100 that can be configured with a first magnet 710 having a first magnetic pole 711, a second magnetic pole 712, and a midpoint 713, and a second magnet 720 having a first magnetic pole 721, a second magnetic pole 722, and a midpoint 723, and a ferromagnetic conductor 900. The first magnet 710 and second magnet 720 can be straight bar magnets offset from the pivot point 200 and positioned such that the second magnetic pole 712 of the first magnet 710 meets the first magnetic pole 721 of the second magnet 720 such that the first magnet 710 and second magnet 720 form an angle that opens toward the pivot point 200. The ferromagnetic conductor 900 can be located where the first magnet 710 and second magnet 720 meet. The first magnet 710, second magnet 720, and ferromagnetic conductor 900 can be configured to produce a slightly asymmetric magnetic field that is substantially coaxial with the pivot point 200 of the hub 103 to be monitored by a magnetic field sensor 303.

FIG. 18 depicts an example of a hub 103 of an orthopedic brace 100 that can be configured with a first magnet 710 having a first magnetic pole 711, a second magnetic pole 712, and a midpoint 713. The first magnet 710 can be a straight bar magnet configured to be offset from the pivot point 200 and perpendicular to a radius 1500 of the hub 103. The first magnet 710 can be configured to produce a magnetic field to be monitored by a magnetic sensor 303.

FIG. 19 depicts an example of a hub 103 of an orthopedic brace 100 that can be configured with a first magnet 710 having a first magnetic pole 711, a second magnetic pole 712, and a midpoint 713. The first magnet 710 can be a straight bar magnet configured to be offset from the pivot point 200 and colinear with a radius 1500 of the hub 103. The first magnet 710 can be configured to produce a magnetic field to be monitored by a magnetic sensor 303.

FIG. 20 depicts an example of a hub 103 of an orthopedic brace 100 that can be configured with a first magnet 710 having a first magnetic pole 711, a second magnetic pole 712, and a midpoint 713, and a second magnet 720 having a first magnetic pole 721, a second magnetic pole 722, and a midpoint 723 and a ferromagnetic conductor 900. The first magnet 710 and second magnet 720 can be embedded in the hub 103 on opposing sides of the pivot point 200 such that the first magnetic pole 711 and second magnetic pole 712 of the first magnet 710 are divided along a plane perpendicular to the axis of rotation and the first magnetic pole 721 and second magnetic pole 722 are also divided along a plane perpendicular to the axis of rotation and such that first magnetic pole 711 of the first magnet 710 and the second magnetic pole 722 of the second magnet 720 face the same direction. The ferromagnetic conductor 900 can be configured to connect the first magnet 710 and second magnet 720 such as by connecting the first magnet 710 and second magnet 720 with a disk shaped ferromagnetic conductor 900, a ring shaped ferromagnetic conductor 900, a “C” shaped ferromagnetic conductor 900, or other path shaped ferromagnetic conductor 900. The first magnet 710, second magnet 720, and ferromagnetic conductor 900 can be configured to produce a magnetic field to be monitored by a magnetic field sensor 303.

### External Attachments

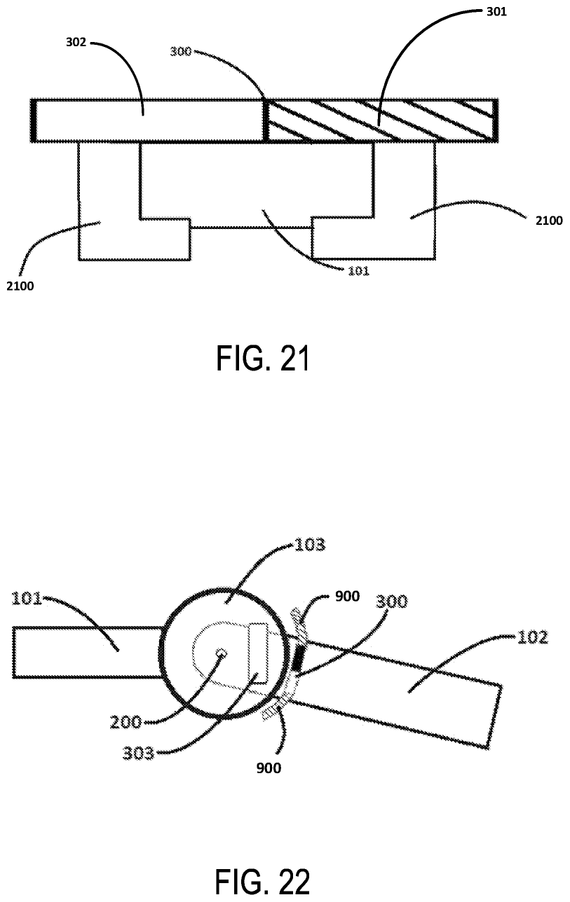

An orthopedic brace 100 can be configured to include a magnet 300 that can be externally affixed to the orthopedic brace 100. FIG. 21 shows a cross section of an example of an external magnet affixed to a brace member with a clip. The orthopedic brace 100 can include a magnet 300 and a clip 2100 or other attachment configured to externally affix the magnet 300 to a first brace member 101 such that the magnet 300 is positioned where the operation of the brace joint produces magnetic field rotation about a location in a second brace member 102 proximal to the magnetic field sensor 303. The magnet 300 can be configured to be externally affixed to the orthopedic brace 100 such as by using a clip, adhesive, screw, or other means. The magnet 300 can be retained in a non-ferromagnetic housing to help protect the magnet 300.

FIG. 22 depicts an example of a bracing portion of an orthopedic brace 100 configured with an external magnet. Generally, in an example of the orthopedic brace 100 an externally affixed magnet outside of the hub 103 area can attract contaminants and can be subject to mechanical interference. In an example of the orthopedic brace 100 it can be desirable for an external magnet to be low profile; it can be desirable for an external magnet 300 to be near the magnetic field sensor 303. External magnets can get in the way, may be knocked off, and can collect debris. However, external magnets can offer advantages such as providing longer pole separation, being removable, and helping to retain the strength of a brace member. An external magnet 300 can be longer than the width of a brace member. An external magnet 300 can include a ferromagnetic flux conductor 900, which can help provide longer effective pole separation that can be advantageous. External magnets can be configured to be easily attachable, detachable, or removable. Removable magnets can help improve ease of cleaning and can be replaceable or retrofitted to existing braces.

### Attachment Adapters

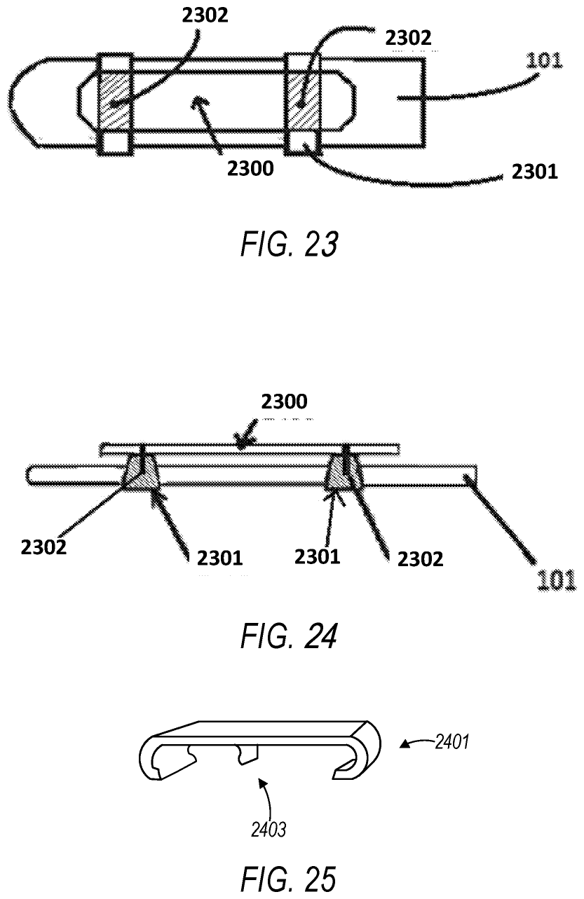

FIG. 23 and FIG. 24 show an example of the orthopedic brace 100 that can include an added function board 2300, affixed with an attachment adapter such as an adapter clip 2301, and a fixation pin 2302. The orthopedic brace member 101 can include a mechanical coupling component configured with two interfaces, a first interface, configured to engage one or more structures of the orthopedic brace 100 such as using an interface fit, adhesion, retention pin, or screw, and a second interface, configured to mechanically couple to the added function board. The mechanical coupling component can be configured to support an added function board 2300, an external magnet 300, an aesthetic cover 2600, or other component.

An orthopedic brace 100 can include an attachment adapter that can be used to connect the orthopedic brace 100 to elements to help enhance functionality such as an element configured for angle detection, activity monitoring, connectivity, memory, display, power, or aesthetic improvement. An attachment adapter can help to couple common additive elements, such as an angular range of motion sensor, an added function board 2300, or an aesthetic attachment, to a variety of brace configurations.

FIG. 25 shows a perspective view of an example of a portion of an attachment adapter such as an adapter clip 2301. The adapter clip 2301 can be configured as a “C” shaped clip that grabs the first brace member 101 or second brace member 102, such that the adapter clip 2301 can hold an added function board 2300 in place. The added function board 2300 can be affixed to the adapter clip 2301. The added function board 2300 may be affixed using a screw, adhesion, retention pin, interface fit, or other means.

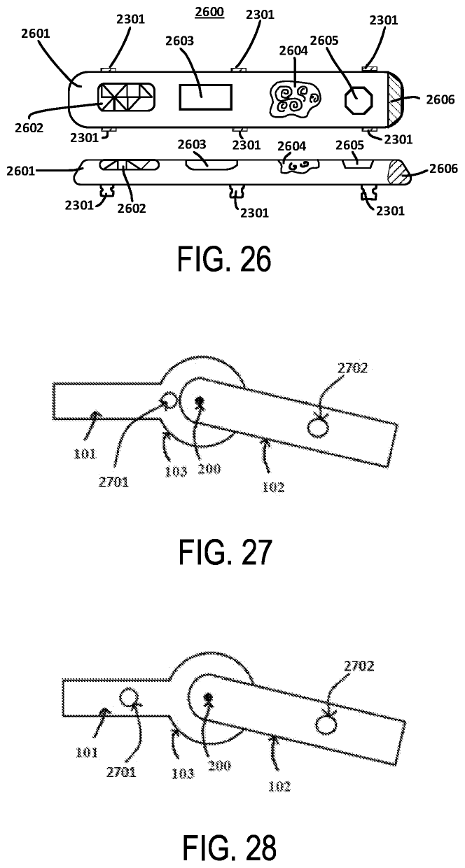

FIG. 26 shows perspective views of an example of an aesthetic cover 2600 that may include an attachment adaptor such as one or more attachment point or adapter clip 2301 and a decorative bezel 2601. The decorative bezel 2601 may include one or more aesthetic features such as a light display window 2602, an open port 2603, an artistic decoration 2604, a sensor or actuator port 2605, a focused light lens 2606, or other aesthetic feature.

An added function board 2300 can include one or more of several elements of the system. The dimensions and characteristics of an element can be generally consistent or uniform across a range of target brace designs. An attachment adapter may help allow a common element to be attached to various brace elements and styles. An adapter may be configured to accommodate an aesthetic cover 2600, which can help shield the added function board 2300 from damage. An aesthetic cover 2600 can include a lens, an optical filter, an image, or a human interface element such as a display or touch control.

### Gyroscopes

FIG. 27 and FIG. 28 depict an example of a bracing portion of an orthopedic brace 100 that can be configured to use gyroscopic data to determine the angular position of the orthopedic brace 100. Gyroscope data can help determine the angle between the first brace member 101 and the second brace member 102, across the pivot point 200, by comparing a signal from a first gyroscope 2701 affixed to the first brace member 101, and a signal from a second gyroscope 2702 affixed to the second brace member 102.

An equation or function may be used to compare multi-axis data such as when the first gyroscope 2701, the second gyroscope 2702, and the pivot point 200, do not define a plane orthogonal to the rotational axis.

A comparative function can be applied to the signals from the first gyroscope 2701 and second gyroscope 2702, where the plane of the cross section defines an X-Y plane. An axis of rotation signal Ω can be used for an axis of rotation that is coaxial with the rotational axis of the orthopedic brace 100. The first gyroscope 2701 can be labeled ΩH, and the second gyroscope 2702 (can be labeled ΩA. An example of an expression for the change of angle across a short time interval ΔΩ(t₁-t₂) can be {(ΩH₂−ΩA₂)−(ΩH₁−ΩA₁)} which can account for the pivot point 200 angle change and discount any change in the common orientation of the orthopedic brace 100 over the time interval.

An absolute angle of the pivot point 200 can be calculated, where the absolute angle of the pivot point measures the angle formed between the first brace member 101 and the second brace member 102 about the pivot point 200. An example calculation of absolute angle θ can be: θ=θ₀−Σ(ΔΩ) where θ₀ is the starting angle of the orthopedic brace 100 and ΔΩ is the change of angle of the orthopedic brace 100.

A practical way of setting the starting angle θ₀ can include circuitry configured to automatically enter a specific value of θ₀ when the system is powered on.

A practical way of setting the starting angle θ₀ can include circuitry configured to enter a limit value stored in memory when the orthopedic brace 100 reaches a mechanical limit.

A practical way of setting the starting angle θ₀ can include circuitry configured to automatically enter a starting angle when the system detects a signal coupled to a fixed relationship in the assembly. The signal can be a switch, vibration, a derivative of the angle signal, an acceleration, an optical signal, or other signal. The signal can alternatively, or additionally, be manually triggered.

Automatic operation, such as when automatically entering a starting angle θ₀ at a mechanical limit, a fixed relationship, or when receiving a signal, can include circuitry configured for continuous, single, or multiple angle correction. Such as entering a starting angle at both extremes of motion of the orthopedic brace 100 or entering an angle at multiple fixed angle triggers. Continuous angle corrections can help compensate for gyroscopic sensor drift.

Gyroscopic sensor drift may result from a gyroscopic incorrectly reporting low levels of motion when the gyroscope is not in motion, which can accumulate into significant errors over time. An orthopedic brace 100 can include circuitry configured to compensate for gyroscopic drift. The circuitry can be configured to compensate for gyroscopic drift using several approaches, singly or in combination.

An approach to compensate for gyroscopic drift can include algorithmically ignoring Ω values or ΔΩ values below some number €.

An approach to compensate for gyroscopic drift can include enforcing a θ₀ signal to occur within a time window before allowing a new ΔΩ calculation, such as requiring that θ₀ be no older than a specified value, such as 100 seconds, (T<Emax/Average Drift) which can help prevent invalid data from being reported.

An approach to compensate for gyroscopic drift can include measuring an average drift over time and subtracting the average drift estimate from the gyroscope signal to calculate the individual gyroscopic sensor drift values, which can help extend intervals of operation without a valid θ₀ update.

An approach to compensate for gyroscopic drift can include enforcing short intervals of θ₀ positioning to collect data on average drift values over time ΔΩd. ΔΩd can be subtracted to compensate for gyroscopic drift.

### Example Embodiment

FIG. 3 shows a side or cross-sectional view of an example of portions of an orthopedic brace 100 and a system to monitor the range of motion of the orthopedic brace. An orthopedic brace 100 can include a first brace member 101, a second brace member 102, a pivot point 200, a hub 103, and an angular range of motion sensor that can include a magnet 300 and a magnetic field sensor 303. The first brace member 101, and the second brace member 102, of the orthopedic brace 100 can be a structural element configured to couple to musculoskeletal aspects of a body and connected at the pivot point 200 with a hub 103 formed about the pivot point 200. FIG. 3 shows an example of the orthopedic brace 100 configured such that the pivot point 200 can include an axle about which the first brace member 101 and the second brace member 102 can rotate such that the hub 103 acts as a hinge for the orthopedic brace 100.

An example of the angular range of motion sensor can include a magnet 300 affixed to the first brace member 101 that can define a magnetic field that can be monitored by a magnetic field sensor 303 affixed to the second brace member 102. The relation of the magnetic field to the magnetic field sensor 303 is variable with the angular motion of the orthopedic brace 100. The relation can be used to calculate an angular range of motion for an angle that describes the relationship between the first brace member 101 and the second brace member 102 about a vertex located at the pivot point 200 of the orthopedic brace 100. The magnetic field sensor 303 can include a single-axis magnetic field sensor 303, or a multi-axis magnetic field sensor, which can produce at least a first signal, a second signal, or a combination of signals.

An example of the magnetic field sensor 303 can include compensation circuitry configured to adjust for a secondary component of variation in the magnetic field produced by a magnet 300. A secondary component of variation in the magnetic field can occur if the central axis of the magnetic field is not substantially coaxial with the rotational axis of the orthopedic brace 100.

Compensation circuitry can be configured to linearize a non-linear distribution such as one created by a secondary component of variation in a magnetic field monitored by a magnetic field sensor 303 through the range of angular motion of the orthopedic brace 100. The compensation circuitry can be configured to adjust for a secondary component of variation using a calibration data set. The compensation circuitry can be configured to use an interpolation between the values of the data set to compensate for a secondary component of variation. The compensation circuitry can fit a calibration data set to an equation to adjust for a secondary component of variation in the magnetic field 300.

An example of the orthopedic brace 100 may include a multi-axis magnetic field sensor 303, and compensation circuitry configured to use an equation or function such as a two argument arctan function to process the first signal and second signal, from the multi-axis magnetic field sensor, to produce a result. The result can be used in combination with an interpolation between values of a calibration data set such as to compensate for a secondary component of variation in the magnetic field. The result can be used in combination with an equation, to which a calibration data set has been fitted, such as to adjust for a secondary component of variation in the magnetic field.

FIG. 27 and FIG. 28 show a diagram of an example of an orthopedic brace 100 configured to monitor the range of motion of the orthopedic brace. The orthopedic brace 100 can include a include a first brace member 101, a second brace member 102, a pivot point 200, a hub 103, and an angular range of motion sensor. The angular range of motion sensor can include at least a first gyroscope 2701 that produces a first signal and a second gyroscope 2702 that produces a second signal. The first gyroscope 2701 can be affixed to the first brace member 101 of the orthopedic brace 100, such as depicted in FIG. 27, The first gyroscope 2701 can be affixed to the hub 103 of an orthopedic brace 100, such as depicted in FIG. 28. The second gyroscope 2702 can be affixed to the second brace member 102. The first gyroscope 2701 and second gyroscope 2702 can be a single-axis gyroscope, or a multi-axis gyroscope. The angular range of motion sensor can include compensation circuitry that can be configured to use the first signal and the second signal to determine the relative angular motion of the orthopedic brace 100, which can be used to determine the absolute angle of the orthopedic brace 100. The orthopedic brace 100 can include circuitry configured to compensate for gyroscopic drift.

The orthopedic brace 100 can include a memory location that can store an indication of a starting angle that can be used as a reference for measuring the angular motion of the orthopedic brace 100. The orthopedic brace 100 can include circuitry and features to collect or enter the starting angle of the orthopedic brace 100. The orthopedic brace 100 can include circuitry configured to automatically enter a stored indication of a starting angle when the system is powered on. The orthopedic brace 100 can include circuitry configured to enter a stored indication of a starting angle when the pivot point 200 reaches a defined angle limit.

### Additional Configurations

An orthopedic brace 100 can include a mechanical structure with flexural elements, stiff elements, structural elements configured to couple to the musculoskeletal aspects of the body, an angular measurement subsystem configured to observe flexural, rotational, or translational changes between structural elements. An angular measurement subsystem can be configured to communicate to a microprocessor for data storage or integration with additional physiological environmental, or internal signals for later retrieval or for conversion into immediate or delayed auditory, visual, tactile, electronic, or photonic information.

An orthopedic brace 100 can be configured with additional sections such as with additional joints or brace members, such as an orthopedic brace 100 that includes a first brace member connected to a second brace member by a first joint and a third brace member connected to the second brace member by a second joint.

An orthopedic brace 100 can include padded elements such as to help provide additional comfort for a patient.

An orthopedic brace 100 can include a pre-existing brace connected to additional elements of the brace system. An orthopedic brace 100 can include a measurement subsystem integrated with structural elements. An orthopedic brace 100 can include a power source that can include a rechargeable or replaceable battery.

An orthopedic brace 100 can include a sensing mechanism that evaluates whether the system is being worn by the user or tracks the don and doff history of the device over time, or both.

An orthopedic brace 100 can include one or more panels or covers to conceal or to highlight the functional structures of the system. For example, the panels and covers can be formed to allow access to control and information from the system to be viewed by the user. The panels and covers can be configured to diffuse light and sound. The panels and covers can be configured to be easily detachable by the user and exchanged for alternate versions of the component such as for aesthetic pleasure or functional advantage.

An orthopedic brace 100 may be configured to generate a variety of outputs such as to provide information or pleasure to a user of the system. For example, the outputs can include one or more of visible lights, sounds, infrared signals, vibrations, music, laser beams, magnetic fields, or vapors.

An orthopedic brace 100 can be configured to receive as inputs a variety of signals including one or more of touch, pressure position, time, sound, vibration, relative position, joint angle, posture, visible light, infrared light, temperature, linear acceleration, and magnetic fields.

A monitoring system for an orthopedic brace 100 can include a system of attachments or components configured to provide additional functionality to a non-augmented orthopedic brace 100. The system of attachments or components can include elements such as a magnet, a display panel, an aesthetic cover 2600, a magnetic field sensor 303, compensation circuitry, a battery, a gyroscope, or a combination of multiple elements. The attachments or components can be configured to augment a specific class of brace, such as by using attachments configured with an adapter clip 2301, or other attachment adapter, configured to attach to a specified brace element such as a brace element of a specific class or model or produced by a specific manufacturer.

An orthopedic brace 100 may be configured to produce an output such as displays of light, sound, vibration, music, and various outputs in response to combinations of sensor inputs, programmed parameter thresholds, or time.

An orthopedic brace 100 can be configured so that the feedback stimuli (light, sound, vibration, etc.) produced by the system are modulated by one or more of the following signal data: the angle of flexion, angular velocity, angular acceleration, duration of angular velocity, duration of motion, number of identifiable excursions, excursions per unit time, pattern matching of motion parameters to target template parameters, linear acceleration, sensor angle relative to gravity, derivative of excursion rate, EGM signals, user operated switches and sensors.

An orthopedic brace 100 can be configured such that the system can be enabled with wireless connectivity to a remote device such as for readout or programming capability.

An orthopedic brace 100 can be configured so that the system can use wired or wireless connections such as to allow additional display or other output or input capabilities.

An orthopedic brace 100 can be configured so that the system can employ a remote readout with wireless connectivity that may connect to remote monitors, computers, or television displays for mirrored or augmented display outputs including graphs, videos, sounds, images, or other display outputs such as to entertain, inform, or motivate.

An orthopedic brace 100 can be configured so that the system can couple to one or more external devices for extending the range of sensory and exercise combinations to include weight training machines, sports equipment such as hockey sticks, golf clubs, tennis racquets, bats, swords, nets, balls, elastic trainers, spring trainers, kettle bells, treadmills, cross trainers, or physical therapy or other training equipment, etc.

An orthopedic brace 100 can be configured so that the system can couple physical or physiological data to one or more ancillary external devices such as for tracking, control, intensity, duration, frequency, and repetition recordings and interactive controls and signaling.

An orthopedic brace 100 can be configured so that one, some, or all system components are configured to attach to existing braces using clips, straps, bands, clamps, pins, screws, rivets, snaps, fabric, tethers, or adhesives.

An orthopedic brace 100 can be configured so that one, some, or all the system components can be integrated with a brace system.

An orthopedic brace 100 can be configured to preferentially fit with brace systems with matching mechanical coupling, electrical wires, magnets, or fitment features that provide improved ease of adding the system elements to braces created for extensibility to active braces.

An orthopedic brace 100 can be configured so that the system can employ one or more magnets mounted to one or more elements of a brace and a magnetic sensor mounted to one or more elements of a brace to provide a mode of detecting relative motion, angular, linear, or relative to the environment.

An orthopedic brace 100 can be configured so that the system can employ one or more gyroscopic sensors such as to provide angular acceleration, velocity, position, or impulse signals to provide a mode of detecting relative motion, angular, linear, or relative to the environment.

An orthopedic brace 100 can be configured so that the system can employ one or more acceleration sensors such as to provide angular acceleration, velocity, position, or impulse signals to provide a mode of detecting relative motion, angular, linear, or relative to the environment.

An orthopedic brace 100 can be configured so that the system can employ one or more externally located (off the brace) sensors such as to determine angular acceleration, velocity, position, or impulse signals to provide a mode of detecting relative motion, angular, linear, or relative to the environment.

An orthopedic brace 100 can be configured so that one or more programmable gesture modes can allow selectable motions or position combinations such as to trigger selectable visual, audible, electronic, vibratory, electromagnetic, or photonic events.

An orthopedic brace 100 can include one or more integrated game modes that can stimulate repeated enjoyment or execution of behavioral sequences, such as for providing gamified physical therapy by communicating pivot angle information to one or both of a gaming controller console or a gaming controller handheld user interface.

An orthopedic brace 100 can include computational processes to convert one or more signals into corrected angular or linear motion parameters such as angle in degrees or radians and the various derivatives and integrals of these signals.

An orthopedic brace 100 can be configured with an audio amplifier and audio speaker, such that the noises of the system can include, but are not limited to “ray guns”, animal noises, squeaks, engines, robot voices, laughter, applause, cheering, vehicles, spaceships, explosions, radio noises, beeps, whistles, sirens, horns, harps, instruments, chords, drums, user recordable sounds, etc.

An orthopedic brace 100 can be configured so that the system can employ one or more sensors to provide proximity information, the sensors being configured to sense one or more of light, magnetic fields, radio signals, acoustic signals, or vibrations such as for modulating the system functionality in a specific location or spatial relation to other devices or environmental factors.

An orthopedic brace 100 can be configured so that the system or some system functions can be remotely activated, deactivated, or modulated by external environments or remote devices such as for the purpose of programming, initiating, terminating, or pausing functions of the system.

An orthopedic brace 100 can be configured so that the system can receive programming changes or report system or user data via light, magnetic fields, radio signals, acoustic signals, or vibrations.

An orthopedic brace 100 can be configured to couple to another active brace system for one or more coordinated device behaviors, such as can include one or more of guided exercise, game play, or coaching, such as by providing data on an athlete's motion, such as throwing a ball or swinging a bat or golf club, when an athlete is training.

An orthopedic brace 100 can be configured to be paired or coupled with external devices such as exercise or physical therapy equipment or an environmental beacon such as to provide contextual interaction with the environment and conditioned recording of the activities or limitations of the brace system's functional behavior.

An orthopedic brace 100 can be configured to be used as an interactive aesthetic element, such as for a toy or robot costume or as a wearable user-input device for a Virtual Reality (VR) system, by itself, or using multiple such orthopedic braces 100, or using one or more orthopedic braces in combination with one or more other VR user-input devices. A VR user input device can translate user position or movement information into action within a VR environment, such as to move or position an avatar in the VR environment at least in part based on the input received from the orthopedic brace 100 or other VR user input device.

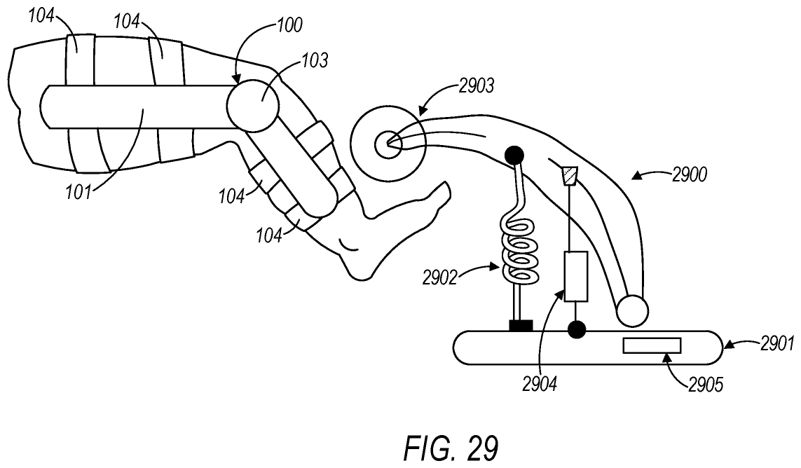

FIG. 29 depicts an example of an orthopedic brace 100 that can be paired or coupled with one or more external devices, such as exercise, physical therapy, or training equipment 2900. For example, the external training equipment 2900 can include a base 2901, a weight or resistance element 2902, and a moving element 2903. The training equipment 2900 may also include at least one of a sensing element 2904 and communication element 2905, such as to record and transmit data on the use of the exercise equipment or other environmental data. For example, data recorded and transmitted by the training equipment 2900 can include information about an amount of weight being lifted by the user of the training equipment 2900 wearing the orthopedic brace 100, an amount of resistance being applied to the training equipment 2900 being used by the user of the training equipment 2900 wearing the orthopedic brace 100, a range of motion being applied to the training equipment 2900 being used by the user of the training equipment 2900 wearing the orthopedic brace 100, a number of repetitions performed, or the like.

Data collected by the external training equipment 2900 can be communicated to a user interface device for the orthopedic brace 100, where it can be stored and used in coordination with range-of-motion or other data from the orthopedic brace 100. For example, range of motion data from the orthopedic brace 100 can be combined with RFID or other data from a free weight or other piece of exercise or training equipment 2900 being used by the wearer of the orthopedic brace 2900, such as to identify the amount of weight being lifted or the amount of resistance being applied, and, therefore, the amount of work being performed during the exercise, therapy, or training session. In this way, data collected by the orthopedic brace 100 can be weighted by environmental or other data collected by an external device, such as the exercise or training equipment 2900.

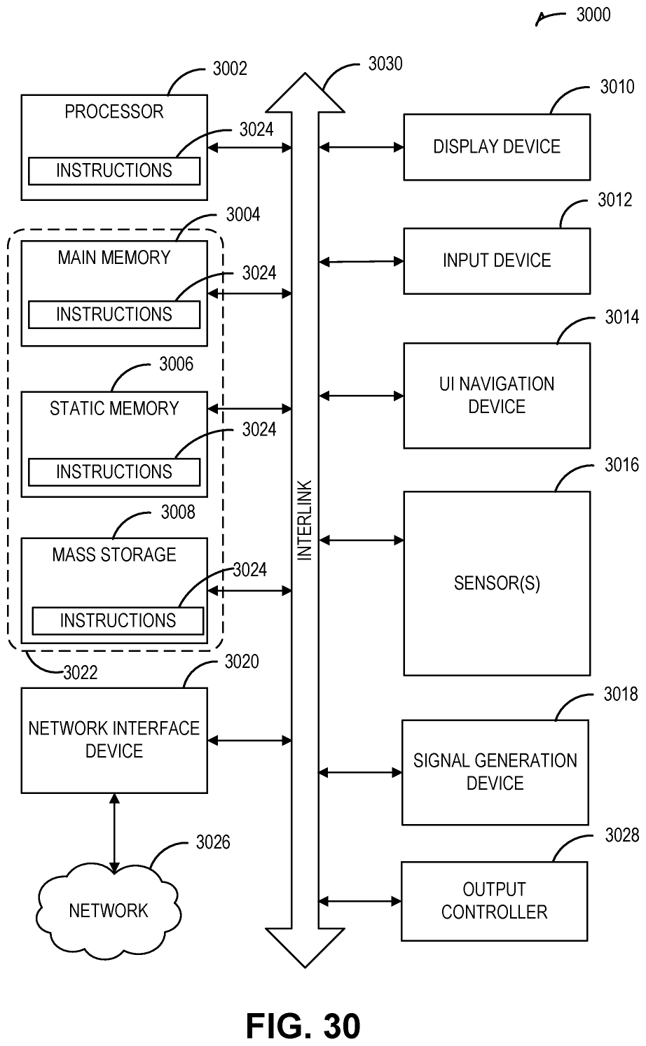

FIG. 30 is a block diagram of an example of an apparatus, device, or machine 3000 upon which any one or more of the techniques (e.g., methodologies) discussed herein may perform. The machine 3000 may operate as a standalone device or can be connected (e.g., networked) to other machines. The machine 3000 can be connected to a sensor 3016, such as can include any of the pivot angle sensors disclosed herein. Such connection can be wireless, such as from a Bluetooth or other wireless transceiver that can be included in or coupled to the pivot angle sensor. In a networked deployment, the machine 3000 may operate in the capacity of a server machine, a client machine, or both in server-client network environments. In an example, the machine 3000 may act as a peer machine in peer-to-peer (P2P) (or other distributed) network environment. The machine 3000 can include a personal computer (PC), a tablet PC, a set-top box (STB), a personal digital assistant (PDA), a mobile telephone, a web appliance, a network router, switch or bridge, a gaming controller (e.g., console, handheld, or both), or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. A circuit set is a collection of one or more circuits that can be implemented in tangible entities that can include hardware (e.g., electrical circuitry, gates, logic, etc.). Circuit set membership can be flexible over time and underlying hardware variability. Circuit sets can respectively include one or more members that can, alone or in combination, perform one or more specified operations when operating. For example, hardware of the circuit set can be immutably configured to carry out a specific operation (e.g., hardwired). The hardware of the circuit set may include switchably or other variably connected physical components (e.g., execution units, transistors, electrical circuits, etc.) that can include a computer readable medium that can be physically modified (e.g., magnetically, electrically, moveable placement of invariant massed particles, etc.) to encode instructions of the specific operation. In connecting the physical components, the underlying electrical properties of a hardware constituent can be changed, for example, from an insulator to a conductor or vice-versa. The instructions can enable embedded hardware (e.g., the execution units or a loading mechanism) to create members of the circuit set in hardware via the variable connections to carry out portions of the specific operation when in operation. Accordingly, the computer readable medium can be communicatively coupled to the other components of the circuit set such as when the device is operating. In an example, any of the physical components can be used in more than one member or of more than one circuit set. For example, during operation, one or more execution units can be used in a first circuit of a first circuit set at a first time and re-used by a second circuit in the first circuit set, or by a third circuit in a second circuit set at a different second time.

The machine 3000 (e.g., a computer system) can include a hardware processor 3002 (e.g., a central processing unit (CPU), a graphics processing unit (GPU), a hardware processor core, field programmable gate array (FPGA), or any combination thereof), a main memory 3004 and a static memory 3006, some or all of which may communicate with each other or with one or more other components via an interlink (e.g., bus) 3030. The machine 3000 can further include or be coupled to a display device 3010, an alphanumeric or other input device 3012 (e.g., a keyboard), and a user interface (UI) navigation device 3014 (e.g., a mouse, a handheld gaming controller remote, or the like). In an example, the display device 3010, input device 3012, and the UI navigation device 3014 can include a touch screen display. The machine 3000 may additionally include a storage device 3008 (e.g., memory circuitry, hard drive, or the like), an audio or other signal generation device 3018 (e.g., a speaker), a network interface device 3020 connected or connectable to a network 3026, and one or more sensors 3016, such as a global positioning system (GPS) sensor, compass, accelerometer, gyroscope, magnetic field sensor, orthopedic brace pivot angle sensor, or other sensor. The machine 3000 may include an output controller 3028, such as a serial (e.g., universal serial bus (USB), parallel, or other wired or wireless (e.g., infrared (IR), near field communication (NFC), etc.) connection to communicate or control one or more peripheral devices (e.g., a printer, card reader, gaming controller console, gaming controller handheld user interface device, etc.).

The storage device 3008 may include a machine readable medium 3022 on which can be stored one or more sets of data structures or instructions 3024 (e.g., software) embodying or used by any one or more of the techniques or functions described herein. The instructions 3024 may also reside, completely or at least partially, within the main memory 3004, within static memory 3006, or within the hardware processor 3002 during execution or performance thereof by the machine 3000. One or any combination of the hardware processor 3002, the main memory 3004, the static memory 3006, or the storage device 3008 may constitute machine readable media.

While the machine readable medium 3022 is illustrated as a single medium, the term “machine readable medium” may include a single medium or multiple media (e.g., a centralized or distributed database, and/or associated caches and servers) configured to store the one or more instructions 824. The term “machine readable medium” may include any non-transitory medium that is capable of storing, encoding, or carrying instructions for execution by the machine 800 and that cause the machine 800 to perform any one or more of the techniques of the present disclosure, or that is capable of storing, encoding, or carrying data structures used by or associated with such instructions. Non-limiting machine readable medium examples may include solid-state memory and optical and magnetic media. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An orthopedic brace system, comprising:
a first brace member, arranged to be externally affixed to a first musculoskeletal portion of a subject;
a second brace member, arranged to be externally affixed to a second musculoskeletal portion of the subject;
a pivot point, affixing the first brace member to the second brace member to permit angular motion therebetween and about a rotational axis defined by the pivot point; and
an angular range of motion sensor, arranged configured to measure an angular motion of the orthopedic brace along an angular range of motion of the pivot point about the rotational axis defined by the pivot point, the angular range of motion sensor including:
a magnetic field sensor and a magnet, arranged to indicate the angular range of motion by sensing a magnetic field of the magnet that varies with the angular motion of the pivot point.

2. The orthopedic brace system of claim 1, wherein the magnet is affixed to one of the first brace member or the second brace member to define the magnetic field that varies with the angular motion of the pivot point, and wherein the magnetic field sensor is affixed to at least one of the first brace member or the second brace member, and wherein at least one of:
(a) the magnet defines a fixed magnetic field central axis that passes through the rotational axis of the pivot point throughout the angular range of motion of the pivot point; or
(b) the magnetic field sensor includes or is coupled to compensation circuitry configured to adjust for a secondary component of variation in the magnetic field through a range of the angular motion of the pivot point.

3. The orthopedic brace system of claim 2, wherein the compensation circuitry is configured to linearize a non-linear distribution of the secondary component of variation in the magnetic field through the range of angular motion of the pivot point.

4. The orthopedic brace system of claim 2, wherein the magnetic field sensor is a single axis magnetic field sensor.

5. The orthopedic brace system of claim 2, wherein the compensation circuitry is configured to adjust for a secondary component of variation in the magnetic field using an interpolation between values of a calibration data set.

6. The orthopedic brace system of claim 2, wherein the compensation circuitry is configured to adjust for a secondary component of variation in the magnetic field using a calibration data set fitted to an equation.

7. The orthopedic brace system of claim 2, wherein the magnetic field sensor is multi-axis sensor that produces at least a first signal and a second signal.

8. The orthopedic brace system of claim 7, wherein the compensation circuitry is configured to process the first signal and the second signal using a two argument arctan function to produce a result.

9. The orthopedic brace system of claim 8, wherein the compensation circuitry is configured to adjust for a secondary component of variation in the magnetic field using the result in combination with an interpolation between values of a calibration data set.

10. The orthopedic brace system of claim 8, wherein the compensation circuitry is configured to adjust for a secondary component of variation in the magnetic field using the result in combination with an equation to which a calibration data set is fitted.

11. The orthopedic brace system of claim 1, wherein the angular range of motion sensor includes a wireless transceiver configured to communicate with a handheld or console portion of a gaming controller to use angular range of motion data for gamified physical therapy.

12. An orthopedic brace system, comprising:
a first brace member, arranged to be externally affixed to a first musculoskeletal portion of a subject;
a second brace member, arranged to be externally affixed to a second musculoskeletal portion of the subject;
a pivot point, affixing the first brace member to the second brace member to permit angular motion therebetween and about a rotational axis defined by the pivot point;
an angular range of motion sensor, configured to measure the angular motion of the pivot point along an angular range of motion of the pivot point about the rotational axis defined by the pivot point, the angular range of motion sensor including:
a first gyroscope that produces a first signal, affixed to the first brace member; and
a second gyroscope that produces a second signal, affixed to the second brace member.

13. The orthopedic brace system of claim 12, wherein the first gyroscope is a multi-axis gyroscope, the second gyroscope is a multi-axis gyroscope, and the pivot point is configured with one or more axis of rotation.

14. The orthopedic brace system of claim 12, wherein the angular motion is measured as a relative angular motion determined using the signal of the first gyroscope and the signal of the second gyroscope.

15. The orthopedic brace system of claim 14, further comprising memory location storing an indication of a starting angle used as a reference for measuring the angular motion of the pivot point relative to the starting angle used as the reference.

16. The orthopedic brace system of claim 15, further comprising circuitry configured to automatically enter the stored indication of a starting angle when the system is powered on.

17. The orthopedic brace system of claim 15, further comprising a mechanical feature defining an angle limit of the pivot point and circuitry configured to automatically enter the stored indication of a starting angle when the pivot point reaches the defined angle limit.

18. The orthopedic brace system of claim 15, further comprising circuitry configured to automatically enter the stored indication of a starting angle when a signal is detected.

19. The orthopedic brace system of claim 15, further comprising circuitry configured to enter the stored indication of starting angle when manually triggered.

20. The orthopedic brace system of claim 15, further comprising circuitry configured to compensate for gyroscopic drift.
